(19) United States
(12) Patent Application Publication
(10) Pub. No.: US 2004/0176613 A1
(43) Pub. Date: Sep. 9, 2004

(54) Method for preparing n-($g(v)-bromoalkyl)phthalimides

(76) Inventors: Jean-Philippe Gillet, Brignais (FR); Chirstophe Ruppin, Saint-Pierre D'Albigny (FR)

Correspondence Address: Marcy M Hoefling, Albemarle Corporation, 451 Florida Street, Baton Rouge, LA, 70801-1765, US

(21) Appl. No.: 10/490,887

(22) PCT Filed: Sep. 26, 2002
(86) PCT No.: PCT/FR02/03274

(30) Foreign Application Priority Data
Sep. 27, 2001 (FR) 01/12421

(51) Int. Cl. C07D 209/48

(52) U.S. Cl. 548/473

(57) ABSTRACT

The invention concerns a procedure for preparation of N-(ω-bromoalkyl)phthalimides which consists of reacting potassium phthalimide with an α,ω-dibromoalkane without cosolvent, at a temperature from 50° C. to 130° C. by using a molar ratio of α,ω-dibromoalkane to potassium phthalimide from 3 to 8, of cooling the reaction medium to about 80° C.-85° C., of washing it with water, of decanting, of removing excess α,ω-dibromoalkane from the organic phase under reduced pressure, and then of crystallizing the N-(ω-bromoalkyl)phthalimide with a low boiling point alcohol.

The present invention concerns a procedure for preparation of N-(ω-bromoalkyl)phthalimides with formula:

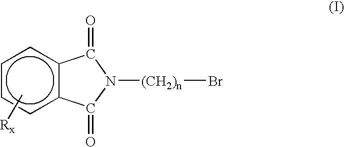

in which n is a whole number from 2 to 8, R represents a hydrocarbon radical, straight-chain or branched, with 1 to 4 carbon atoms, a halogen atom, or an alkoxy radical, x is equal to 0, 1, 2, 3 or 4 and when x=2, 3 or 4, the R radicals may be identical or different.

These compounds are intermediates in the synthesis of pharmaceutical products such as antibiotics or neuroleptics.

The method most currently used for preparing compounds with formula (I) consists of reacting potassium phthalimide (II) with an α,ω-dibromoalkane (III) according to reaction (1):

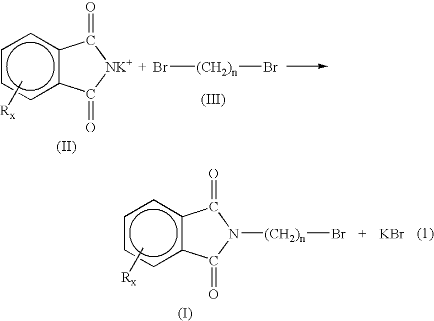

The potassium salt (II) may be used as is. It may also be prepared in situ generally by the action of potassium carbonate (anhydrous) or potash with the phthalimide with formula:

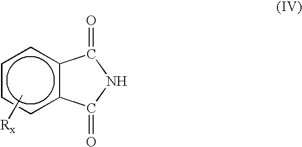

In the patent, U.S. Pat. No. 5,541,230, a procedure for preparation of N-(4-bromobutyl)phthalimide is described that consists of reacting 30 mmol of potassium phthalimide with 100 mmol of 1,4-dibromobutane in 50 mL of DMF. The reaction medium is stirred for 2 h at 60° C. and then the DMF and the excess 1,4-dibromobutane are evaporated under reduced pressure. The remaining residue is extracted with CHCl₃ and water. The compound obtained is crystallized with ethanol.

H. FORD et al. (J. Am. Chem. Soc., 103, 26, pp. 7773-7779, 1981) describes a procedure for preparation of N-(6-bromohexyl)phthalimide according to the following method.

77 g of potassium phthalimide (0.416 mol) are introduced into a solution of 253 g of 1,6-dibromohexane (1.04 mol) in 250 mL of DMF.

The reaction medium, brought to 90° C. under stirring, is kept under stirring and at this temperature for 75 min.

Next, the DMF and excess 1,6-dibromohexane are removed under reduced pressure (6 mmHg) at 90° C.-130° C.

The solid residue is dissolved in 50 mL of CHCl₃ and the mixture, filtered if necessary, is extracted with 2 times 75 mL of water, washed with 40 mL of a solution of 0.1 molar sodium carbonate in order to remove the potassium phthalimide and washed again with 40 mL of water.

The CHCl₃ is removed under reduced pressure and the residue is triturated with 300 mL of ether and the solution is filtered. The diphthalimidohexane (17 g) is rejected. The ether is removed from the filtrate.

The solid residue is recrystallized with 250 mL of 75% ethyl alcohol and then dried under vacuum.

The N-(6-bromohexyl)phthalimide is obtained at a yield of 66%.

These procedures present the drawback of using three solvents; especially DMF for condensation, CHCl₃ for the extraction of the product from the reaction medium and ethanol for carrying out the purification.

T. A. Crabb et al. (J. Chem. Soc. Perkin Tran. I, pp 191-195 (1985)) obtained N-(4-bromobutyl)phthalimide with a yield of 32% by carrying out the condensation of potassium phthalimide with 1,4-dibromobutane used according to a molar ratio of dibrominated compound/potassium salt equal to 1.35 in acetone by heating at reflux for 24 h. The KBr is removed by filtration and the solvent is evaporated. The obtained product is purified by successive recrystallizations with petroleum ether.

In patent application EP 330 625, N-(3-bromopropyl)phthalimide is obtained with a yield of 72% according to the following method.

In a 1-L round-bottomed flask provided with stirring and cooling means are introduced 101 g (0.5 mol) of 1,3-dibromopropane, 250 mL of acetone and 15 g of potassium phthalimide. The reaction medium is brought to reflux under stirring to which are added at intervals of 1 h, 15 g, 10 g and 6.3 g of potassium phthalimide (or a total of 46.3 g: 0.25 mol). It is kept at reflux 24 h in all. Next the KBr is removed by filtration and the acetone is evaporated.

The oil obtained is distilled under reduced pressure to remove the excess 1,3-dibromopropane and then the N-(3-bromopropyl)phthalimide is recrystallized twice in ethanol to remove the diphthalimidopropane, product of dicondensation.

Generally, the procedures that we have just described present the drawback of using 2, even 3 solvents that are either very flammable (acetone) or toxic (DMF) and in addition, these procedures require numerous operations for purification of the products.

P. L. Salzberg and J. V. Supniewski (Organic Syntheses, Coll. Volume 1, Second edition, John Wiley and Sons Inc. 1941, New York, pp. 119-121) describe the preparation of β-bromoethylphthalimide according to the following method.

Into a 1-L round-bottomed flask equipped with effective stirring and a reflux condenser, are introduced 150 g of potassium phthalimide (0.81 mol) and 450 g of 1,2-dibromoethane (2.4 mol) with a boiling temperature of 129° C./131° C.

Stirring is begun and the mixture is heated approximately 12 h by means of an oil bath kept at a temperature of 180/190° C.

Next, the excess dibromoethane is removed under reduced pressure. 290 to 295 g of 1,2-dibromoethane are recovered.

The crude product is separated from the KBr by dissolving in 300 mL of 98/100% alcohol brought to reflux until the black oil is entirely solubilized.

The hot ethanolic solution is filtered, and the KBr cake obtained is washed with hot ethanol. The alcohol of the ethanolic filtrate is distilled under reduced pressure and then the dry residue is treated at reflux with 500 mL of CS₂ for approximately 50 min in order to separate the soluble β-bromoethylphthalimide from the insoluble diphthalimidoethane. The suspension is hot filtered and the CS₂ is removed under reduced pressure.

The β-bromoethylphthalimide obtained with a yield of 70-80% is presented in the form of yellow-brown crystals melting at 78-80° C.

To obtain a pure product, it is necessary to recrystallize the crude product with dilute alcohol in the presence of decolorizing activated charcoal.

R. H. Mizzoni et al. (J.A.C.S. Vol. 76, p. 2416, 1954) adapted this method to prepare N-(4-bromobutyl)phthalimide that they obtained with an overall yield of 61% after treatment of the mother liquors.

The drawback of the latter procedures is the use of reaction temperatures that are too high and that create by-products and colorations of desired products requiring numerous purification operations with flammable solvents (CS₂, cyclohexane) and decolorization operations.

The applicant has found a simple preparation procedure for compounds with formula (I) which consists of condensing potassium phthalimide (II) with an α,β-dibromoalkane (III) without use of cosolvents and then of extracting and purifying the product obtained with the minimum of operations without using toxic and/or very flammable solvents.

Therefore, the invention has as a goal a procedure for preparation of N-(ω-bromoalkyl)phthalimides with formula:

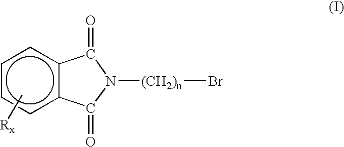

in which n is whole number from 2 to 8, and preferably, from 3 to 6, R represents a hydrocarbon radical, straight-chain or branched, with 1 to 4 carbon atoms, a halogen atom such as Cl or Br, or an alkoxy radical such as CH₃O—, x is equal to 0, 1, 2, 3 or 4 and when x=2, 3 or 4 the R radicals may be identical or different, which consists of reacting potassium phthalimide (II) with an α,ω-dibromoalkane (III) according to reaction (1):

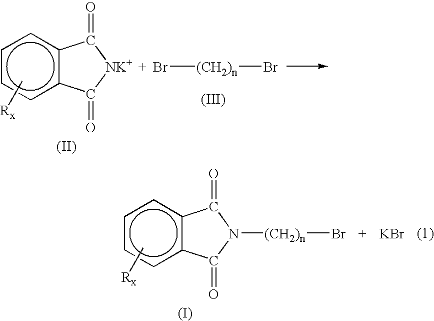

characterized in that it consists of carrying out the condensation reaction (1) of potassium phthalimide (II) with α,ω-dibromoalkane (III) without cosolvent, under stirring at a temperature from 50° C. to 130° C. and preferably, from 100 to 120° C., by using a molar ratio of α,ω-dibromoalkane (III) to potassium phthalimide (II) from 3 to 8 and, preferably from 4 to 6, then the reaction terminated, the reaction medium is cooled from about 80° C. to 85° C., at least one washing of said reaction medium is carried out with sufficient amounts of water to solubilize all the KBR formed, it is decanted, then the excess α,ω-dibromoalkane is removed under reduced pressure from the organic phase obtained, and the N-(ω-bromoalkyl)phthalimide obtained is crystallized with a low boiling point alcohol and it is dried under reduced pressure at a temperature equal to 50° C., at most.

According to the present invention, an alcohol whose boiling point is equal to 100° C. at most is designated as a low boiling point alcohol.

Methanol, ethanol, isopropanol, n-propanol, tert-butanol, and 2-butanol will be mentioned by way of illustration of usable alcohols according to the present invention.

Preferably, ethanol or isopropanol will be used.

According to the present invention, two to three washings are carried out and preferably 150 to 300 g of water in total are used per mole of potassium phthalimide.

The condensation reaction (1) is carried out generally at atmospheric pressure, under an inert gas atmosphere such as nitrogen.

The reaction duration may vary to a great extent. The end of the reaction is determined by measurement of the KBr formed.

Generally, the reaction time is the time necessary to entirely transform the potassium phthalimide. It is a function of the stirring, the temperature and the molar ratio (III)/(II). The ratio influences the by production of products of dicondensation with formula:

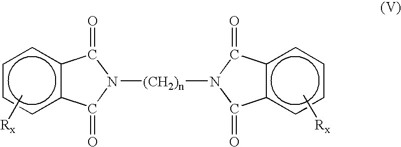

A molar ratio (III)/(II) less than 3 causes difficult stirring of the reaction medium and favors the formation of said products of dicondensation (V).

According to the present invention, after removal of the excess α,ω-dibromoalkane from the organic phase, said organic phase is taken up by a low boiling point alcohol brought to reflux and then left to cool under stirring at room temperature. The N-(ω-bromoalkyl)phthalimide that crystallizes is recovered by cold filtration; the cake is washed with the previously mentioned alcohol and then dried under reduced pressure at a temperature equal to 50° C. at most.

The procedure according to the present invention applies particularly well to the preparation of N-(ω-bromoalkyl)phthalimides with formula (I) in which x=0 and n is from 3 to 6.

The purity of the products obtained is sufficient to allow their use directly in later syntheses.

The following example illustrates the invention.

### EXAMPLE

### Synthesis of N-(4-bromobutyl)phthalimide with a 1,4-dibromobutane/potassium Phthalimide Ratio of 5

Into a Sovirel type reactor mechanically stirred, provided with a condenser, an opening for introduction of solids, a system for inerting with nitrogen, and a temperature probe, are loaded 5 mol (1081 g) of 1,4-dibromobutane and 1 mol (188 g; 98.5% pure) of potassium phthalimide. The reaction medium is brought under stirring to 115° C. and then kept at this temperature for 15 h. It is verified that the conversion is complete by a measurement of the potassium bromide in the reaction medium.

After cooling of the reaction medium to 80° C., 200 g of water are added. After 15 min of stirring and then decantation, the aqueous phase containing the potassium bromide is removed and then a second washing is carried out (still at 80° C.) with 50 g of water.

The ascending condenser is then replaced by a distillation column and then the excess 1,4-dibromobutane is distilled under reduced pressure (8-10 mbar) with a bottom temperature from 80 to 120° C. Thus, 815 g of 1,4-dibromobutane are recovered corresponding to a recovery rate of 1,4-dibromobutane of 94%. This latter with a purity greater than 99.8% may be directly recycled into a later operation.

At the end of the distillation, the reaction medium is left to cool to 75-80° C. and then 325 g of ethanol are added. After homogenization and obtention of a clear organic solution at reflux of ethanol, the reaction medium is left to cool under stirring to room temperature (˜20° C.).

After crystallization of the N-(4-bromobutyl)phthalimide, the ethanolic suspension of the product is filtered on frit at room temperature. The moist cake is washed with 35 g of ethanol and then dried at 50° C. under reduced pressure (20 mmHg). Thus, 268 g of N-(4-bromobutyl)phthalimide are obtained that correspond to a molar yield of 92% compared with the potassium phthalimide provided.

1. Procedure for preparation of N-(ω-bromoalkyl)phthalimides with formula: 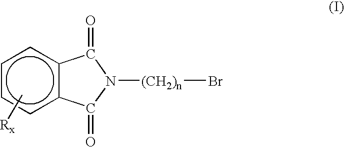
in which n is a whole number from 2 to 8, and preferably, from 3 to 6, R represents a hydrocarbon radical, straight-chain or branched, with 1 to 4 carbon atoms, a halogen atom such as Cl or Br, or an alkoxy radical such as CH₃O—, x is equal to 0, 1, 2, 3 or 4 and when x=2, 3 or 4 the R radicals may be identical or different, which consists of reacting potassium phthalimide (II) with an α,ω-dibromoalkane (III) according to reaction (1): 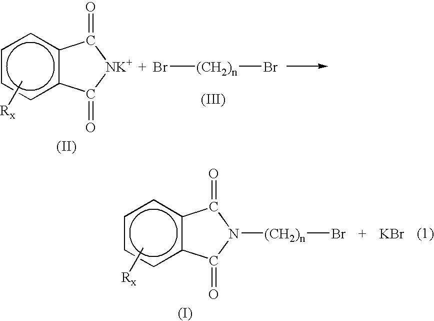
characterized in that it consists of carrying out the condensation reaction (1) of the potassium phthalimide (II) with the α,ω-dibromoalkane (III) without cosolvent, under stirring at a temperature from 50° C. to 130° C., by using a molar ratio of α,ω-dibromoalkane (III) to potassium phthalimide (II) from 3 to 8, then the reaction terminated, the reaction medium is cooled with sufficient amounts of water to solubilize all the kbr formed, it is decanted, then the excess α,ω-dibromoalkane is removed under reduced pressure from the organic phase obtained, and the N-(ω-bromoalkyl)phthalimide obtained is crystallized with a low boiling point alcohol and it is dried under reduced pressure at a temperature equal to 50° C. at most.

2. Procedure according to claim 1 characterized in that the temperature at which the condensation reaction (1 ) is carried out is from 100° C. to 120° C.

3. Procedure according to claim 1 or 2 characterized in that the molar ratio of α,ω-dibromoalkane (III) to potassium phthalimide (II) is from 4 to 6.

4. Procedure according to any one of claims 1-3 characterized in that the alcohol with low boiling point has a boiling point equal to 100° C. at most.

5. Procedure according to claim 4 characterized in that the alcohol with low boiling point is methanol, ethanol, isopropanol, n-propanol, tert-butanol or 2-butanol.

6. Procedure according to claim 5 characterized in that the alcohol with low boiling point is isopropanol or ethanol.

7. Procedure for preparation of N-(ω-bromoalkyl)phthalimides with formula (I) according to any one of claims 1 to 6 in which x=0 and n is from 3 to 6.

8. Procedure according to claim 7 in which n is equal to 4.
